(19) United States
(12) Patent Application Publication
(10) Pub. No.: US 2012/0295940 A1
(43) Pub. Date: Nov. 22, 2012

(54) COMPOSITIONS FOR TREATING MASTITIS

(75) Inventors: Burt R. Sookram, Palm Harbor, FL (US); John W. Veenstra, Plano, TX (US)

(73) Assignee: Noble Ion, LLC, Plano, TX (US)

(21) Appl. No.: 13/295,882

(22) Filed: Nov. 14, 2011

Related U.S. Application Data
(60) Provisional application No. 61/413,456, filed on Nov. 14, 2010.

(51) Int. Cl. A61K 31/4425; A61P 29/00; A61K 31/14

(52) U.S. Cl. 514/358; 514/643

(57) ABSTRACT

The present disclosure relates to compositions that are effective in controlling or in preventing mastitis in an domesticated animal. The disclosed compositions comprise a biocidal system, comprising a primary biocide and a pH buffer component; a skin conditioner and moisturizer; a cationic or ionic surfactant having an HLB of from about 5 to about 30; an emollient system comprising an extradermal penetrating agent and an emollient base; a thickening agent; and an aqueous based carrier.

### RELATED U.S. APPLICATION

This application claims priority to U.S. Provisional Application No. 61/413,456 filed Nov. 14, 2010, the entire disclosure of which is incorporated herein by reference.

### FIELD OF THE INVENTION

The present invention relates to compositions that are effective in controlling or in preventing mastitis in domesticated animals. More specifically, the invention pertains to compositions for treating mastitis rapidly and without negative effects to the treated skin. Even more specifically, the invention pertains to compositions for treating mastitis that incorporate a biocidal system, a surfactant, a skin conditioner, an emollient system, a thickening agent and a carrier.

### BACKGROUND OF THE DISCLOSURE

Bovine mastitis is an inflammation of the udder. Milk from cows suffering from mastitis has an increased somatic cell count. This condition, which is almost exclusively initiated by pathogenic microorganisms that have entered the teat canal after the milking process, occludes milk flow and production, and can permanently impair a domesticated animal's future ability to produce milk.

The rate of new udder infection is related to the number of mastitis-causing pathogens on teat ends. Disinfecting teats with a germicidal agent immediately after milking kills most of the pathogens on teats. This in turn reduces the chance of those pathogens getting into the udder.

Post-milking teat disinfection is especially effective against the contagious pathogens Staphylococcus aureus and Streptococcus agalactiae. While milking can spread any type of mastitis pathogen, these two pathogens in particular spread from cow to cow during the milking process. Post-milking teat disinfection is less effective in reducing the new infection rate of “environmental” pathogens such as coliforms and Streptococcus species other than Streptococcus agalactiae. Control of environmental pathogens requires management practices including maintaining cows in a clean, dry environment, good pre-milking hygiene, including pre-milking teat disinfection and thoroughly drying teats; and using functionally adequate milking machines. Typically, milkers should continue post milking teat disinfecting as a routine part of milking procedures, even if Streptococcus agalactiae has been eliminated and somatic cell counts are low.

The usual sources of harmful microorganisms include an unsanitary stable/pen environment, unsanitary milking equipment, the milking personnel, cross contamination for other mastitic domesticated animals, and the domesticated animal's own elimination (defecation/urination) processes. It is estimated that each year hundreds of millions of dollars are lost to this disease in the United States alone. Estimates of total annual milk product lost in the United States due to mastitis range as high as 40 percent. It has been estimated that mastitis' costs about $200 per cow per year and the reduction in milk production accounts for about 70% of the total loss associated with mastitis.

Somatic cells are a normal constituent of milk and only when they become excessive do they indicate a problem. Somatic cells are composed of leucocytes (75%) and epithelial cells (25%). Leucocytes (white blood cells) increase in milk in response to infection or injury while the increase in epithelial cells is the result of infection or injury. The number of cells reflects the severity of mastitis. Somatic cells are expressed either as cells/ml or as SCC of milk. High counts are considered abnormal and indicate possible infections. To be used for human consumption, milk must have less than 750,000 SCC. Milk markets rely routinely on SCC to help ensure good quality milk. Bulk Tank SCC is an indicator of the herd's udder health status.

Recently it has been concluded by the U.S. National Mastitis Council that the use of a pre-milking sanitization step further decreases mastitis, and presents other benefits, such as decreasing the surface pathogen load (such as Escherichia coli and Listeria spp.) and pathogen-related toxin content of milk. Therefore, the industrial recommendation for the use of teat sanitizers presently involves both a pre- and post-milking application. The presently-recommended process of milking is therefore as follows: prior to milking, the teats of the domesticated animal to be milked are sanitized with the pre-milking sanitizer, which is then quickly wiped off with a clean towel. The domesticated animal is then milked with the automated milking machines. After milking, the teat is highly susceptible to infection, because the milk canal and teat-tip sphincter muscle (responsible for closing the teat-end) remains open for approximately 30 minutes after milking. Therefore, a post-milking sanitizer is applied and left on the skin (i.e. not rinsed off or deliberately removed) until the next milking.

While there are a number of germicides that are effective in preventing and treating mastitis, most preparations have the disadvantage of only remaining in contact with the udder for a short time due to the mobility of the preparation. Longer contact time is desirable in order to insure a higher kill rate for the harmful bacteria or faster acting biocides are required. This can be achieved by using a product that is a cream, gel, spray, dip or a foam.

Because the teat sanitizer is left on the skin for a long period of time, the formulation must not have a tendency to irritate or damage the skin. Any toxic effects would be even more pronounced in a typical four-a-day schedule for a milking herd, where the pre- and post-milking sanitization applications could reach up to eight times per day. Due to the difficulty in formulation of a composition which has a satisfactory antimicrobial activity but which also does not damage the skin, the majority of compositions exist in the field that are indicated for use as either a pre-milking, biocidal sanitizer, or as a post-milking biocidal sanitizer/skin conditioner. Generally, the pre-milking sanitizers contain a lower germicidal activity (usually a lower concentration of biocidal active ingredients) than post-milking sanitizers/conditioners because the pre-milking sanitizer does not remain in prolonged contact with the skin and the milk canal are not yet open so infection rates are lower.

Typical active ingredients for teat sanitizer compositions include iodine; although, others have been used. Iodine is perhaps the most widely used active ingredient in such compositions, mainly due to its low cost and fairly broad antimicrobial spectrum. At concentrations allowable in milk, however, iodine has a relatively slow kill time in comparison to other popular active agents. Iodine also confers no persistence of antimicrobial activity (i.e. continued killing ability due to retention of the active ingredient in the target tissue) with continued use. Furthermore, at concentrations necessary for usefulness as a biocidal agent, iodine damages the udder skin in frequent milking situations and may not be compatible with other active antimicrobial agents used at other steps in the milking process. Even in once- to twice-daily milking situations, iodine can have a long-term negative effect on the udder skin condition, due to tissue denaturation, and to the formation of salts of the counter-ion with environmental anions (e.g., Cl—) on the skin surface after the product has dried on the teat.

There is therefore a long felt need for compositions for treating mastitis in an domesticated animal that has a rapid kill time, is compatible with the skin of the domesticated animal being treated and which has no long-term negative effects on the udder skin condition. The compositions disclosed herein meet these and other needs.

### SUMMARY OF THE INVENTION

The disclosed compositions provide for preventing and controlling mastitis in an domesticated animal. The compositions and methods are suitable for use with any domesticated animal, including, but not limited to, cows, goats, sheep, and the likes.

Accordingly a primary objects of the invention is providing compositions effective in killing one or more pathogens, non-limiting examples of which include Pseudomonas aeruginosa, Staphylococcus aureus, Staphylococcus epidermidis, Streptococcus agalactiae, Brucella melitensis, Corynebacterium bovis, Mycoplasma, Escherichia coli, (E. coli) and Klebsiella pneumoniae.

A further object of the invention is providing compositions effective in preventing the infection and/or spread of one or more pathogens from an infected domesticated animal to other domesticated animals or an apparatus that contacts an infected domesticated animal, for example, milking machines, bedding, stalls, and the like. Non-limiting examples of infection causing pathogens include: Streptococcus agalactiae, Staphylococcus aureous, and Mycoplasma spp.

A further object of the invention is providing compositions effective against environmental pathogens, non-limiting examples of which include Streptococcus spp, Escherichia coli, Klebsiella species, A. pyogenes, and Pseudomonas species. In addition, the disclosed compositions are suitable for controlling yeast.

An additional object of the invention is providing compositions that are a replacement for iodine-based treatments.

Further, an additional object of the invention is providing methods for preventing mastitis in a domesticated animal and methods for treating mastitis in a domesticated animal.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE FIGURES

FIG. 1, is a photograph depicting cracked, dry, infected teats.

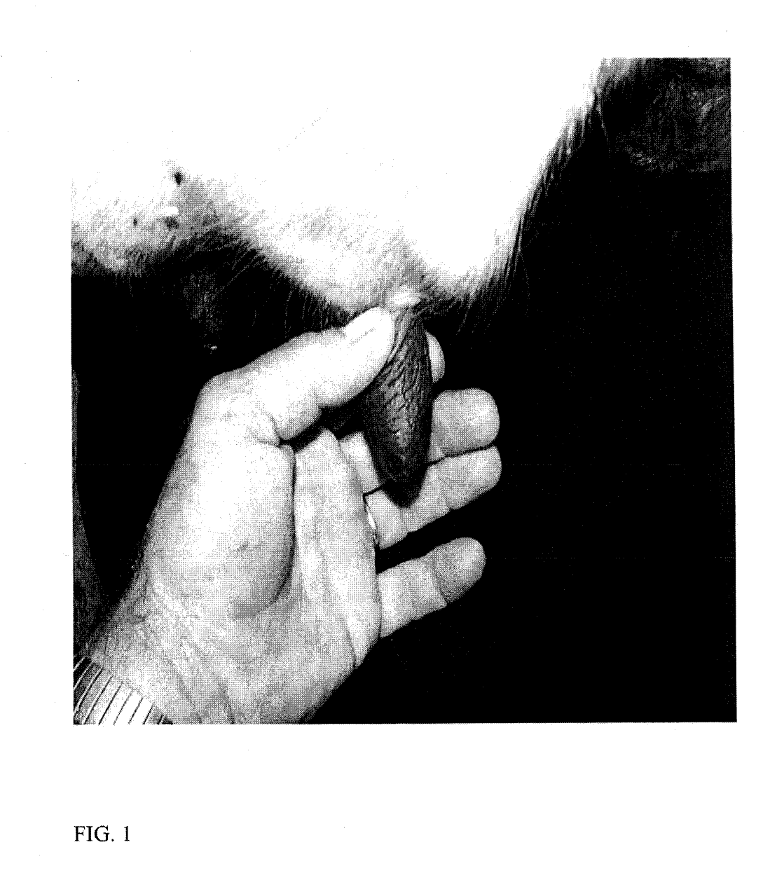

FIG. 2, is a photograph depicting soft, supple, healthy teats after only 14 days of treatment with the compositions disclosed herein.

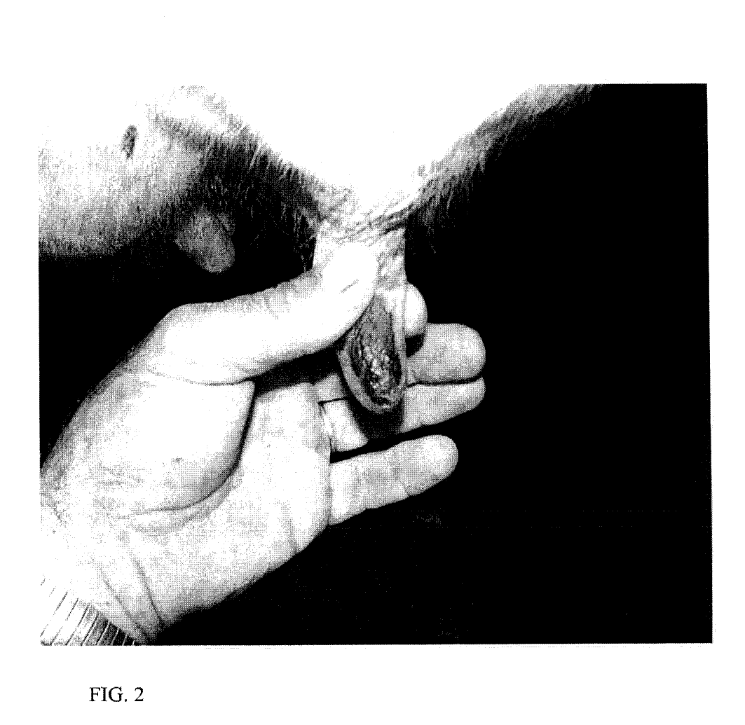

### DETAILED DESCRIPTION AND EMBODIMENTS OF THE INVENTION

The following is a detailed description of the primary components of the invention:

1. Biocidal System

The disclosed compositions comprise a biocidal system. The biocidal system comprises a primary biocide and a pH buffer component. The pH buffer is chosen for compatibility with the primary biocide.

a. Primary Biocide

Suitable biocides include quaternary ammonium compounds chosen from (C₁₂-C₁₄ alkyl)(C₁-C₂ dialkyl)benzyl ammonium salts, N—(C₁₂-C₁₈ alkyl)heteroaryl ammonium salts, and N—[(C₁₂-C₁₄ alkyl)(C₁-C₂ dialkyl)]heteroarylalkylene ammonium salts. Non-limiting examples of the (C₁₂-C₁₄ alkyl)(C₁-C₂ dialkyl)benzyl ammonium salts include (C₁₂-C₁₄ alkyl)dimethyl-benzyl ammonium chloride, (C₁₂-C₁₄ alkyl)dimethylbenzyl ammonium bromide, and (C₁₂-C₁₄ alkyl)dimethylbenzyl ammonium hydrogen sulfate. Non-limiting examples of the N—(C₁₂-C₁₈ alkyl)heteroaryl ammonium salts include cetyl pyridinium chloride, cetyl pyridinium bromide, and cetyl pyridinium hydrogen sulfide. For the N—(C₁₂-C₁₈ alkyl)heteroaryl ammonium salts other anions can be used.

Further examples of quaternary ammonium compounds suitable for use as the primary biocides include cetyltrimethylammonium chloride, stearyltrimethylammonium chloride, isostearyltrimethylammonium chloride, lauryltrimethylammonium chloride, behenyltrimethyl-ammonium chloride, octadecyltrimethylammonium chloride, cocoyltrimethylammonium chloride, cetyltrimethylammonium bromide, stearyltrimethylammonium bromide, lauryl-trimethylammonium bromide, isostearyllauryldimethylammonium chloride, dicetyldimethyl-ammonium chloride, distearyldimethylammonium chloride, dicocoyldimethylammonium chloride, γ-gluconamidopropyldimethylhydroxyethylammonium chloride, di-[polyoxyethylene(2)]oleylmethylammonium chloride, dodecyldimethylethylammonium chloride, octyldihydroxyethylmethylammonium chloride, tri[polyoxyethylene(5)]-stearylammonium chloride, polyoxypropylenemethyldiethylammonium chloride, lauryl-dimethyl(ethylbenzyl)ammonium chloride, behenamidopropyl-N,N-dimethyl-N-(2,3-dihydroxypropyl)ammonium chloride, tallowedimethylammoniopropyltrimethylammonium dichloride, and benzalconium chloride.

Other suitable biocides include organic acids which are safe under the FDA GRAS guidelines for food production yet still effective in controlling bacteria, viruses and parasites.

Suitable organic acids are Lactic, Acetic, Formic, Fumaric, Citric, Oxalic, Adipic and Uric.

Other suitable organic acids are the carboxylic acids, whose acidity is associated with their carboxyl group —COOH. Sulfonic acids, containing the group —SO2OH, are relatively stronger acids. The relative stability of the conjugate base of the acid determines its acidity. In some biological systems more complex organic acids such as L-lactic, citric, and D-glucuronic acids are formed. These use the hydroxyl or carboxyl group.

The third group of suitable organic acids are Humic, Sebacic, Stearic, Gallic, Palmitic, Caffeic, Glyoxylic, Fulvic, Carnosic, Anthranilic, Ellagic, Lipoic, Chlorogenic, Rosmarinic, Phosphoric, Methacrylic, Oleanic, Nitrohumic, Florocinnamic, Hexaflorosilicic, Hydrofluoric, Hydroxycitric and Silicofluoric.

The fourth group of suitable organic acids is fruit acids. The acids in fruits are chiefly acetic, malic, citric, tartaric, oxalic, and in some instances boric.

The fifth group of suitable organic acids is beta hydroxy acids which is a type of phenolic acid. Salicylic acid is a colorless crystalline organic acid whose main active ingredient obtained from this source is a monohydroxiybenzoic acid.

The sixth group of suitable organic acids is a class of products that break biofilm. Biofilms are the protective layer/barrier that surround bacteria. Some species are not able to attach to a surface on their own but are often able to anchor themselves to the matrix or the bacteria cells. It is during this colonization that the cells are able to communicate via its quorum sensing ability. Once colonization has begun, the biofilm grows through a combination of cell division and recruitment. The final stage of biofilm formation is known as development and is the stage in which the biofilm is established and may only change in shape and size. The development of a biofilm may allow for an aggregate cell colony to be increasingly resistant. A biofilm's hard protective surface can be broken by Lactobacillus sc Nisin which is produced by fermentation using the bacterium Lactococcus lactis. This is obtained from the culturing of Lactococcus lactis on natural substrates, such as milk or dextrose, and is not chemically synthesized. This is a peptide which is produced by the food grade dairy starter bacterium Lactococcus lactis.

A seventh group of suitable organic acids is natural enzymes. Enzymes are proteins that catalyze chemical reactions and range from just 62 amino acid residues. Typically these are protease, lipase, diastase and cellulase enzymes. Enzymes are usually very specific as to which reactions they catalyze and the substrates that are involved in these reactions. The shape, charge and hydrophilic/hydrophobic nature characterize the enzymes.

Cetylpyridinium chloride is available from Wako Pure Chemical Industries, Ltd.

b. pH Buffer Component

The pH buffer used is a low pH dermal product with the following range of specifications.

A biocidal, dermal, non-corrosive acid composition, having a maximum proton count of 1.5×10̂25, an embodied conductivity range of from 250 mV to 1500 mV and a 0.1% solution of the composition having a pH of under 2.0. The pH buffer component of the present invention can be a highly protonated, supercharched, low pH, non-corrosive composition. By way of example, such a composition disclosed in U.S. Pat. No. 7,824,524, which is incorporated by reference herein in its entirety, and should be understood to be applicable to the present invention. In addition, other biocidal, dermal, non-corrosive acid compositions could be used providing they have a maximum proton count of 1.5×1025, an embodied conductivity range of from 250 mV to 1500 mV and a 0.1% solution of the composition having a pH of under 2.0.

The disclosed compositions of the preferred embodiment comprise from about 0.05% to about 0.75% by weight of a biocidal system, having:

i) at least about 75% by weight of a primary biocide; and

ii) at least about 5% by weight of a pH buffer component.

Another embodiment of the invention has:

i) from about 75% to about 95% by weight of a primary biocide; and

ii) from about 5% to about 25% by weight of a pH buffer component.

Yet another embodiment has:

i) from about 75% to about 90% by weight of a primary biocide; and

ii) from about 10% to about 25% by weight of a pH buffer component.

A yet further embodiment has:

i) from about 80% to about 95% by weight of a primary biocide; and

ii) from about 5% to about 20% by weight of a pH buffer component.

A still further embodiment has:

i) from about 85% to about 95% by weight of a primary biocide; and

ii) from about 5% to about 15% by weight of a pH buffer component

A non-limiting example of a disclosed biocidal system includes:

i) 90% by weight of cetyl pyridinium chloride; and

ii) 10% by weight of pH buffer

Another non-limiting of the disclosed biocidal system includes:

i) from about 80 to about 95% by weight of cetyl pyridinium chloride; and

ii) from about 5% to about 20% by weight of pH buffer.

A yet further non-limiting example of a disclosed biocidal system includes:

i) from about 80 to about 90% by weight of cetyl pyridinium chloride; and

ii) from about 10% to about 20% by weight of pH buffer.

A still further non-limiting example of a disclosed biocidal system includes:

i) from about 75 to about 90% by weight of cetyl pyridinium chloride; and

ii) from about 10% to about 25% by weight of pH buffer.

The biocidal systems disclosed herein can comprise 75%, 76%, 77%, 78%, 79%, 80%, 81%, 82%, 83%, 84%, 85%, 86%, 87%, 88%, 89%, 90%, 91%, 92%, 93%, 94%, or 95% by weight of a primary biocide.

2. Skin Conditioner and Moisturizing Agent

Skin conditioners which have moisturizing properties suitable for use in the disclosed biocidal systems include urea and urea derivatives, for example, imidazolyl urea, hydantoin, dichlorodimethylhydantoin, bromochlorodimethylhydantoin, dibromodimethylhydantoin, aloe vera, panthenol, allantoin, retinyl palmitate, ergocalciferol, imidazolidinyl urea, and biuret. Further examples of skin conditioners include Trilaurin, Triarachidin, Tribehenin, Tricaprin, Tricaprylin, Trierucin, Triheptanoin, Triheptylundecanoin, Triisononanoin, Triisopalmitin, Triisostearin, Trilinolein, Trilinolenin, Trimyristin, Trioctanoin, Triolein, Tripalmitin, Tripalmitolein, Triricinolein, Tristearin, Triundecanoin, Glyceryl Triacetyl Hydroxystearate, Glyceryl Triacetyl Ricinoleate and Glyceryl Stearate Diacetate are referred to as Glyceryl Triesters. The glyceryl triesters are prepared from glycerin and the corresponding fatty acid. For example, Trilaurin is produced from glycerin and lauric acid; Tristearin is produced from glycerin and stearic acid. Many glyceryl triesters, or triglycerides, can be found in domesticated animal and vegetable fats and oils such as tallow, palm-nut and coconut oils

More examples of skin conditioners and moisturizers are silicone based cyclic compounds such as cyclomethicone, hexamethylcyclotrisiloxane octamethylcyclotetrasiloxane, decamethylcyclopentasiloxane and dodecamethylcyclohexasiloxane.

3. Surfactant

The disclosed compositions comprise from about 0.05% to about 5.0% by weight of a cationic surfactant having an hydrophile-lipophile balance (“HLB”) of from about 5 to about 30. One aspect of the disclosed compositions comprises a cationic or ionic surfactant having an HLB of from about 12 to about 18. A further aspect of the disclosed compositions comprises a cationic or ionic surfactant having an HLB of from about 13 to about 16. Another embodiment of the disclosed compositions comprise from about 0.1% to about 4.0% by weight of a cationic or ionic surfactant.

Suitable cationic or ionic surfactants for use in the disclosed compositions include polyoxyethylene C6-C12 alkylphenyl ethers, polyoxyethylene sorbitan tri(C12-C18)-alkanoates, polyoxyethylene sorbitan di(C12-C18)-alkanoates, polyoxyethylene sorbitan mono-, di-, and tri-(C12-C18)-alkanoates, and polyoxyethylene C12-C20 alkyl ethers.

One category of suitable cationic or ionic surfactants for use in the disclosed compositions are the polyoxyethylene C6-C12 alkylphenyl ethers having the formula:

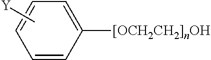

wherein Y is a C6-C12 alkyl unit and n is an index from 5 to 40. Non-limiting examples of C6-C12 alkylphenyl ethers includes polyoxyethylene(5)isooctylphenyl ethers sold under the tradenames IGEPAL™ CA-520 and IGEPAL™ CO-520, polyoxyethylene(8)isooctylphenyl ethers sold under the tradename TRITON™ X-114, polyoxyethylene(9)nonylphenyl ether sold under the tradename IGEPAL™ CO-630, polyoxyethylene(10)isooctylphenyl ether sold under the tradename TRITON™ X-100, polyoxyethylene(branched)nonylphenyl ethers sold under the tradename TRITON™ N-101, polyoxyethylene(12)nonylphenyl ether sold under the tradename IGEPAL™ CO-720, polyoxyethylene(12)isooctylphenyl ether sold under the tradename IGEPAL™ CA-720, polyoxyethylene(40)nonylphenyl ether sold under the tradename IGEPAL™ CO-890, and polyoxyethylene(40)isooctylphenyl ether sold under the tradename TRITON™ X-405.

Another category of cationic or ionic surfactants for use in the disclosed compositions are polyoxyethylene sorbitan mono-, di-, and tri-(C12-C18)-alkanoates, non-limiting examples of which include polyoxyethylene(20) sorbitan trioleate sold under the tradename TWEEN™ 85, polyoxyethylene(20) sorbitan monooleate sold under the tradename TWEEN™ 80, polyoxy-ethylene(20) sorbitan monostearate sold under the tradename TWEEN™ 60, polyoxyethyl-ene(20) sorbitan monopalmitate sold under the tradename TWEEN™ 40, and polyoxyethyl-ene(20) sorbitan monolaurate sold under the tradename TWEEN™ 20.

A further category of cationic or ionic surfactants for use in the disclosed compositions are polyoxyethylene C9-C20 alkyl ethers, non-limiting examples of which include ethoxylate alcohols having the formula:

RO(CH2CH2O)ₘH

wherein R is a linear or branched alkyl group having from 6 to 20 carbon atoms and m is an integer of about 2 to about 20. One example of suitable ethoxylate alcohol surfactants are the NEODOL™ ethoxylated alcohols from Shell Chemicals. Non-limiting examples of suitable ethoxylated alcohols include NEODOL™ 91-5, NEODOL™ 91-6, NEODOL™ 91-8, NEODOL™ 91-9, NEODOL™ 23-6.5, NEODOL™ 25-5, NEODOL™ 25-7, NEODOL™ 25-9, NEODOL™ 25-12, NEODOL™ 45-7, and NEODOL™ 135-7, available from BASF.

4. Emollient System

Emollients are medicinal substances that soften and moisturize the skin. Dry skin occurs as a result of water loss in the top layer of skin. Emollients work by creating an oily layer over the skin, trapping water underneath the surface.

The disclosed compositions comprise from about 1% to about 4% by weight of an emollient system comprising:

i) at least about 20% by weight of an extradermal penetrating agent; and

ii) at least about 50% by weight of an emollient base.

One component of the emollient system relates to extradermal penetrating agents that provide for penetration of dry or damaged teat or utter skin and functions to help carry and retain the biocidal system in contact with the affected tissue. Suitable extradermal penetrating agents include C₁-C₈ mono- or poly-hydroxy alcohols, non-limiting examples of which include benzyl alcohol, ethylene glycol, and propylene glycol. A combination of C1-C8 linear alcohols can also be used as the extradermal penetrating agent, however, the amount of C1-C8 linear alcohol is adjusted according to the type and amount of thickening agent used. This adjustment is within the scope of the artisan. One example of a suitable extradermal penetrating agent is propylene glycol.

The emollient system further comprises an emollient base. The emollient base comprises about one-half of the emollient system. Non-limiting examples of emollient bases includes C9-C14 linear or branched alkyl alcohols, C3-C14 linear or branched polyols, C6-C14 di-esters of C6-C12 diacids, hydrocarbons, natural waxes, vegetable oils, and silicones.

One embodiment of emollient bases includes polyols having the formula:

HOCH₂—[CHOH]ₓ—CH₂OH

wherein the index x is from 1 to 20.

In another iteration of polyols the index x is from 1 to 10. In a further iteration the emollient base includes polyols chosen from glycerol, (2R,3R)-butane-1,2,3,4-tetraol, (2S,3R)-butane-1,2,3,4-tetraol, (2R,3S)-butane-1,2,3,4-tetraol, (2S,3S)-butane-1,2,3,4-tetraol, (2R,3R,4R)-pentane-1,2,3,4,5-pentaol, (2S,3R,4R)-pentane-1,2,3,4,5-pentaol, (2R,3S,4R)-pentane-1,2,3,4,5-pentaol, (2R,3R,4S)-pentane-1,2,3,4,5-pentaol, (2S,3S,4R)-pentane-1,2,3,4,5-pentaol, (2S,3R,4S)-pentane-1,2,3,4,5-pentaol, (2R,3S,4S)-pentane-1,2,3,4,5-pentaol, and (2S,3S,4S)-pentane-1,2,3,4,5-pentaol. In one iteration of the disclosed compositions, the emollient base is glycerol. Various polyols are also known by their common names, inter alia, erythritol and xylitol.

The emollient base can also be a combination of one or more emollient bases, for example, glycerol in combination with ethoxylated partial glyceride fatty acid esters, however, the various other emollient bases that are useful in the present composition include those compatible with the biocidal system and which promote general skin health and integrity in high frequency milking conditions. These include branched chain esters, ethoxylated partial glyceride fatty acid esters, protein derivatives, lanolin and lanolin derivatives, and fatty alcohol ethoxylates, emollient oils, fatty acids, fatty alcohols and their esters. The relative concentrations of extradermal penetrating agent and emollient base in the disclosed compositions are easily determined by those skilled in the art.

A further example of suitable emollient bases include isononyl isonanoate, dioctyl sebacate, isooctyl isooctanoate, dioctyl adipate, squalane, petrolatum, mineral oil, white oil, carnauba wax, candelilla wax, beeswax, sunflower oil, sesame oil, olive oil, lanolin, glycerine, sortibal aloe, poylglycols, polyethylene glycol, polyoxyethylene, polyethylene oxide, cyclomethicone and dimethicone.

In a further embodiment, emollient system comprises from about 1% to about 4% by weight of an emollient system, the emollient system comprising:

i) from about 30% to about 40% by weight of an extradermal penetrating agent; and

ii) from about 60% to about 70% by weight of an emollient base.

In another embodiment, emollient system comprises from about 1% to about 2% by weight of an emollient system, the emollient system comprising:

i) from about 35% to about 45% by weight of an extradermal penetrating agent; and

ii) from about 55% to about 65% by weight of an emollient base.

In a yet further embodiment, emollient system comprises from about 1% to about 2% by weight of an emollient system, the emollient system comprising:

i) from about 25% to about 40% by weight of an extradermal penetrating agent; and

ii) from about 60% to about 75% by weight of an emollient base.

In a still further embodiment, emollient system comprises from about 1% to about 4% by weight of an emollient system, the emollient system comprising:

i) from about 30% to about 40% by weight of an extradermal penetrating agent; and

ii) from about 60% to about 70% by weight of an emollient base.

The compositions disclosed herein can comprise 1%, 2%, 3% or 4% or an emollient system, or any fractional part thereof, for example 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%,

5. Thickening Agent

The disclosed compositions further comprise from about 0.1% to about 4% by weight of a thickening agent. Suitable thickening agents include hydroxynethyl cellulose, hydroxyethyl cellulose, methylcellulose, hydroxypropyl cellulose, methyl cellulose, carboxy methylcellulose, emulsifying waxes, alkyl triammonium methosulfate, and ceteraryl octanoate. Although the disclosed compositions are aqueous based, certain ingredients may require the presence of a more lipophilic solvent for proper stabilization. Preferred additional solvents are polyhydric alcohol solvents, or “polyol” solvents, such as the polyalkylene glycols having alkylene moieties containing about 2-3 carbon atoms, preferably the polyethylene glycols. Molecular weight ranges of from about 200-4000 are preferred for the polyalkylene glycols (e.g., propylene glycol).

Other examples of thickeners are polysaccharides and linear sulfated polysaccharides of natural origin, which increase the viscosity increase in solution, even at small concentrations. These can be classified as uncharged or ionic polymers natural gums obtained from seaweeds. These are Agar, Alginic acid Sodium alginate, Carrageenan (kappa, Iota or lambda), Gum arabic, Gum ghatti, Gum tragacanth, Karaya gum, Guar gum, Locust bean gum, Beta-glucan, Chicle gum, Dammar gum, Glucomannan, Mastic gum, Psyllium seed husks, Spruce gum, Tara gum Gellan gum and Xanthan gum.

Another example of a suitable thickener poylsaccharides is starch which can be unmodified or modified using acid, enzymes, alkaline, bleached, oxidized, acetylated, hydroxpropylated, octenylsuccinic anhydride, carboxyethylated, phosphate, hydroxypropyl, and acetylated oxidated), cationic, cold water, pregelatinized and instant starch.

One embodiment of the disclosed compositions, utilizes hydroxyethyl cellulose in amounts of 0.5%, 0.6%, 0.7%, 0.8%, 0.9%, and 1% by weight of the composition adjusted for the emollient system and for the final method of applying the composition to the domesticated animal in need of treatment.

In a further embodiment, the thickener can be xanthan gum in amounts of 0.5%, 0.6%, 0.7%, 0.8%, 0.9%, and 1% by weight of the composition adjusted for the emollient system and for the final method of applying the composition to the domesticated animal in need of treatment.

6. Carriers

The balance of the disclosed compositions comprises a carrier. The carrier can be any suitable material that can dissolve the active ingredients and co-ingredients and deliver the biocidal system to the infected areas of the domesticated animal being treated. Water is a convenient carrier for liquid embodiments of the disclosed composition. However, alcohols can be used to assist in the dissolving of the ingredients prior to dilution with water. Embodiments of the disclosed compositions include gels, sprays, foams and creams, especially for treating cases wherein the infection may be chronic and the domesticated animal must be isolated from the rest of the domesticated animals and given more intense treatment.

7. Adjunct Ingredients

The disclosed compositions can further comprise one or more dyes at levels of from about 0.001% to 0.5%. Non-limiting examples of suitable dyes are Alizarine Light Blue B (C.I. 63010), Carta Blue VP (C.I. 24401), Acid Green 2G (C.I. 42085), Astrogen Green D (C.I. 42040), Supranol Cyanine 7B (C.I. 42675, Maxilon Blue 3RL (C.I. Basic Blue 80), Drimarine Blue Z-RL (C.I. Reactive Blue 18), Alizarine Light Blue H-RL (C.I. Acid Blue 182), FD&C Blue No. 1 and FD&C Green No. 3. (See U.S. Pat. No. 4,248,827 and U.S. Pat. No. 4,200,606, both incorporated herein by reference).

Other colors which can be Lakes that may be used are FD&C Blue No. 1-Brilliant Blue FCF, (blue shade), FD&C Blue No. 2-Indigotin, (dark blue shade), FD&C Green No. 3-Fast Green FCF, (turquoise shade), FD&C Red No. 40-Allura Red AC, (red shade), FD&C Red No. 3-Erythrosine, (pink shade, commonly used in glacé cherries), FD&C Yellow No. 5-Tartrazine, (yellow shade), FD&C Yellow No. 6-Sunset Yellow FCF, E110 (orange shade)

Another adjunct ingredient suitable for use in the compositions disclosed herein includes fragrances, for example, fragrances as disclosed in U.S. Pat. No. 6,013,618 included herein by reference in its entirety.

A preferred embodiment of the disclosed compositions for improving teat and udder hygiene is as follows:

a) from about 0.05% to about 0.75% by weight of a biocidal system, comprising:
i) at least about 75% by weight of a primary biocide; and
ii) at least about 5% by weight of a pH buffering component;

b) from about 0.05% to about 5.0% by weight of a cationic or ionic surfactant having an

Hydrophilic Lipophilic Balance (HLB) of from about 5 to about 30;

c) from about 1% to about 4% by weight of an emollient system comprising:
i) at least about 20% by weight of an extradermal penetrating agent; and
ii) at least about 50% by weight of an emollient base;

d) a skin conditioner which also has moisturizing properties

e) from about 0.1% to about 4% by weight of a thickening agent; and

f) the balance an aqueous based carrier.

Another embodiment is as follows:

a) from about 0.05% to about 0.75% by weight of a biocidal system, comprising:
i) from about 75% to about 95% by weight of a primary biocide; and
ii) from about 5% to about 25% by weight of a pH buffering component;

b) from about 0.05% to about 5.0% by weight of a cationic or ionic surfactant having an HLB of from about 5 to about 30;

c) from about 1% to about 4% by weight of an emollient system comprising:
i) at least about 20% by weight of an extradermal penetrating agent; and
ii) at least about 50% by weight of an emollient base;

d) a skin conditioner which also has moisturizing properties

e) from about 0.1% to about 4% by weight of a thickening agent; and

f) the balance an aqueous based carrier.

A further embodiment comprises of:

a) from about 0.05% to about 0.75% by weight of a biocidal system, comprising:
i) from about 75% to about 95% by weight of primary biocide; and
ii) from about 5% to about 25% by weight of a pH buffering component;

b) from about 0.05% to about 5.0% by weight of a cationic or ionic surfactant having an HLB of from about 5 to about 30;

c) from about 1% to about 4% by weight of an emollient system comprising:
i) at least about 20% by weight of an extradermal penetrating agent; and
ii) at least about 50% by weight of an emollient base;

d) a skin conditioner which also has moisturizing properties

e) from about 0.1% to about 4% by weight of a thickening agent; and

f) the balance an aqueous based carrier.

A yet further embodiment comprises of:

a) from about 0.05% to about 0.75% by weight of a biocidal system, comprising:
i) at least about 75% by weight of a primary biocide; and
ii) at least about 5% by weight of a pH buffering component;

b) from about 0.05% to about 5.0% by weight of a cationic or ionic surfactant having an HLB of from about 5 to about 30;

c) from about 1% to about 4% by weight of an emollient system comprising:
i) at least about 30% by weight of an extradermal penetrating agent; and
ii) at least about 60% by weight of an emollient base;

d) a skin conditioner which also has moisturizing properties

e) from about 0.1% to about 4% by weight of a thickening agent; and

f) the balance an aqueous based carrier.

However, other non-limiting embodiments and combinations are possible as further disclosed herein.

### Example FORMULATIONS

The following are non-limiting examples of the disclosed compositions:

TABLE I

| Ingredients | 1 | 2 | 3 | 4 | 5 |
|---|---|---|---|---|---|
| cetyl pyridinium chloride | 0.115 | 0.150 | 0.20 | 0.25 | 0.30 |
| pH buffer | 1.0 | 1.0 | 1.0 | 1.0 | 1.0 |
| urea | 0.015 | 0.02 | 0.025 | 0.03 | 0.035 |
| TRITON X-100 | 0.2 | 0.2 | 0.2 | 0.2 | 0.2 |
| PEG 6 | 1.0 | 1.0 | .10 | 1.0 | 1.0 |
| propylene glycol | 1.0 | 1.0 | 1.0 | 1.0 | 1.0 |
| glycerol | 2.0 | 2.0 | 2.0 | 2.0 | 2.0 |
| hydroxyethylcellulose | 0.5 | 0.5 | 0.5 | 0.5 | 0.5 |
| carrier | balance | balance | balance | balance | balance |

TABLE II

| Ingredients | 6 | 7 | 8 | 9 | 10 |
|---|---|---|---|---|---|
| cetyl pyridinium chloride | 0.40 | 0.50 | 0.35 | 0.35 | 0.35 |
| pH buffer | 1.0 | 1.0 | 1.0 | 1.0 | 1.0 |
| urea | — | — | 0.04 | 0.03 | 0.02 |
| PEG 6 | 1.0 | 1.0 | 1.0 | 1.0 | 1.0 |
| TRITON X-100 | 0.2 | 0.2 | 0.2 | 0.2 | 0.2 |
| propylene glycol | 1.0 | 1.0 | 1.0 | 1.0 | 1.0 |
| glycerol | 2.0 | 2.0 | 2.0 | 2.0 | 2.0 |
| hydroxyethylcellulose | 0.5 | 0.5 | 0.5 | 0.5 | 0.5 |
| carrier | balance | balance | balance | balance | balance |

TABLE III

| Ingredients | 11 | 12 | 13 | 14 | 15 |
|---|---|---|---|---|---|
| cetyl pyridinium chloride | 0.35 | 0..35 | 0.35 | 0..35 | 0..35 |
| pH buffer | 1.0 | 1.0 | 1.0 | 1.0 | 1.0 |
| urea | 0.015 | 0.02 | 0.025 | 0.03 | 0.035 |
| TRITON X-100 | 0.2 | 0.2 | 0.2 | 0.2 | 0.2 |
| PEG 6 | 1.0 | 1.0 | 1.0 | 1.0 | 1.0 |
| propylene glycol | 0.5 | 0.75 | 1.0 | 0.5 | 0.75 |
| glycerol | 2.0 | 2.0 | 2.0 | 2.0 | 2.0 |
| hydroxyethylcelluose | 0.5 | 0.5 | 0.5 | 0.5 | 0.5 |
| carrier | balance | balance | balance | balance | balance |

TABLE IV

| Ingredients | 16 | 17 | 18 | 19 | 20 |
|---|---|---|---|---|---|
| cetyl pyridinium chloride | 0.135 | 0.130 | 0.125 | 0.12 | 0.115 |
| pH buffer | | | | | |
| urea | 0.015 | 0.02 | 0.025 | 0.03 | 0.035 |
| TRITON X-100 | 0.2 | 0.2 | 0.2 | 0.2 | 0.2 |
| PEG 6 | 1.0 | 1.0 | 1.0 | 1.0 | 1.0 |
| propylene glycol | 0.5 | 0.75 | 1.0 | 0.5 | 0.75 |
| glycerol | 2.0 | 2.0 | 2.0 | 2.0 | 2.0 |
| hydroxyethylcellulose | 0.1 | 0.25 | 0.4 | 0.75 | 1.0 |
| carrier | balance | balance | balance | balance | balance |

TABLE V

| Ingredients | 21 | 22 | 23 | 24 | 25 |
|---|---|---|---|---|---|
| (C₁₂-C₁₄ alkyl)- | 0.135 | 0.130 | 0.125 | 0.12 | 0.115 |
| dimethylbenzyl | | | | | |
| ammonium chloride | | | | | |
| pH buffer | 1.0 | 1.0 | 1.0 | 1.0 | 1.0 |
| urea | 0.015 | 0.02 | 0.025 | 0.03 | 0.035 |
| TRITON X-100 | 0.2 | 0.2 | 0.2 | 0.2 | 0.2 |
| propylene glycol | 1.0 | 1.0 | 1.0 | 1.0 | 1.0 |
| glycerol | 2.0 | 2.0 | 2.0 | 2.0 | 2.0 |
| Xanthan gum | 0.5 | 0.6 | 0.7 | 0.8 | 0.9 |
| carrier | balance | balance | balance | balance | balance |

TABLE VI

| Ingredients | 26 | 27 | 28 | 29 | 30 |
|---|---|---|---|---|---|
| cetyl pyridinium chloride | 0.135 | 0.130 | 0.125 | 0.12 | 0.115 |
| pH buffer | 1.0 | 1.0 | 1.0 | 1.0 | 1.0 |
| urea | 0.015 | 0.02 | 0.025 | 0.03 | 0.035 |
| TRITON N-101 | 0.2 | 0.2 | 0.2 | 0.2 | 0.2 |
| propylene glycol | 1.0 | 1.0 | 1.0 | 1.0 | 1.0 |
| glycerol | 2.0 | 2.0 | 2.0 | 2.0 | 2.0 |
| Xanthan gum | 1.0 | 1.0 | 1.0 | 1.0 | 1.0 |
| carrier | balance | balance | balance | balance | balance |

The disclosed compositions can be used for various applications with the application route and dosage regimen dictated by the frequency of milking and/or the skin condition of the domesticated animal. As an example of possible applications of the invention, the compositions can be used in domesticated animals as a pre and post-milking application to decrease the potential for mastitis, and/or subcutaneous dermatological pathologies stemming from microbial infections. An example of this includes administering the compositions to skin, specifically the udder and teats of milking domesticated animals. The composition can be applied as a cleanser, scrub (cleanser with abrasive properties), spray, foam, lotion, or gel. The compositions can also be used in a therapeutic manner. For example, the compositions can be used both as a cleanser or a scrub composition to help heal udder and teat skin which has been damaged by frequent milking. Additional applications for the sanitizer include vaginal cleansers, calving sanitizers, burn disinfectants, wound healing aids, and perianal and colostomy wipe applications. For wipes, the formulation of the present invention may be applied to paper or cloth towels.

Although particular dosage regimes may be described in examples herein, a person skilled in the art would appreciated that the dosage regime may be altered to provide optimum therapeutic response. For example, several divided doses may be administered daily or the dose may be proportionally reduced as indicated by the exigencies of the therapeutic situation. In addition, the compositions of the present disclosure can be administered as frequently as necessary to achieve a therapeutic amount.

The following procedure can be used to evaluate the disclosed compositions against various microorganisms. The results below further indicate the effectiveness of the disclosed compositions as measured against state of the art iodine compositions.

A 1% solution of IODINE Teat Dip™ manufactured by AST Inc., Bernville, Pa. 19506 (control) is tested against a 0.1% of the composition disclosed in Example 1.

Materials:

Eschericia coli—ATCC #8739

Staphylococcus aureus—ATCC #6538 Pseudomonas aeruginosa—ATCC #9027

Nutrient agar plates (15 mm×100 mm) # DF0001-17—available from Fisher Scientific.

Incubator 35-37° C.—Precision Scientific Model #6.

A 0.1 mL sample of bacteria was pipetted onto an agar place and uniformly spread across the surface. The inoculum contained from about 1×107 to 1×108 cfu/mL. To one-half of the inoculated plates was charged 15 μl, of the control solution and to the other one-half was charged 15 μL of the composition according to Example 1 from Table I. The plates were then incubated for 24 hours. The amount of inhibition is determined by measuring the size of the zones of inhibition in millimeters using digital calipers. Table A discloses the results of the example procedure described herein.

TABLE A

| Species | Control | Example 1 soln. |
|---|---|---|
| Eschericia coli | 14.22 | 19.18 |
| Staphylococcus aureus | 7.1 | 34.2 |
| Pseudomonas aeruginosa | 15.76 | 20.57 |

Additional testing was completed at BSK Food Laboratory located in Fresno Calif. using E. coli ATCC #25922 strain. The exposure time was 60 seconds and the results are listed in Table B. The reduction in bacterial growth was 99.999%.

TABLE B

| Sample | Control units | Results units |
|---|---|---|
| Biocide 0.15% | 999,000 cfu/ml | <1 cfu/ml |
| Biocide 0.30% | 999,000 cfu/ml | <1 cfu/ml |
| Biocide 0.5% | 999,000 cfu/ml | <l cfu/ml |

*cfu—colony forming units

For a bacterial test using Staphylococcus aureus ATCC #6538 the results are documented in Table C, showing a 99.9999% reduction in bacterial growth.

TABLE C

| Species | Control units | Results units |
|---|---|---|
| Staphylococcus aureus | 7.9 × 10\hat{ }7 cfu/ml | 2.5 cfu/ml |

The next test shows the reduction in the somatic cell count over a 45 day period. Table D reflects the results. The count was reduced from 590,000 to 310,000.

TABLE D

| Dates | Somatic cell count |
|---|---|
| 9/1 | 590.0000 |
| 9/21 | 420,000 |
| 10/11 | 310,000 |

While several embodiments of the present invention have been disclosed hereinabove, it is to be understood that these embodiments are given by example only and not in a limiting sense. Those skilled in the art may make various modifications and additions to the preferred embodiments chosen to illustrate the invention without departing from the spirit and scope of the present contribution to the art. Accordingly, it is to be realized that the patent protection sought and to be afforded hereby shall be deemed to extend to the subject matter claimed and all equivalence thereof fairly within the scope of the invention.

What is claimed is:

1. A composition for improving teat and udder hygiene in domesticated animals comprising:
a) from about 0.05% to about 0.75% by weight of a biocidal system, comprising:
i) at least about 75% by weight of a primary biocide; and
ii) at least about 5% by weight of a pH buffer component;
b) from about 0.05% to about 0.2% by weight of a cationic or ionic surfactant having an HLB of from about 5 to about 30;
c) 0.1% to 5.0% of a skin conditioner
d) from about 1% to about 4% by weight of an emollient system comprising:
i) at least about 20% by weight of an extradermal penetrating agent; and
ii) at least about 50% by weight of an emollient base;
e) from about 0.1% to about 4% by weight of a thickening agent; and
f) an aqueous based carrier.

2. The composition according to claim 1, wherein the primary biocide is a quaternary ammonium salt comprising at least one aryl or heteroaryl unit.

3. The composition according to claim 1, wherein the primary biocide is chosen from (C₁₂-C₁₄ alkyl)(C₁-C₂ dialkyl)benzyl ammonium salts, N—(C₁₂-C₁₈ alkyl)heteroaryl ammonium salts, and N—[(C₁₂-C₁₄ alkyl)(C₁-C₂ dialkyl)]heteroarylalkylene ammonium salts.

4. The composition according to claim 1, wherein the primary biocide is chosen from (C₁₂-C₁₄ alkyl)dimethylbenzyl ammonium chloride, (C₁₂-C₁₄ alkyl)dimethylbenzyl ammonium bromide, (C₁₂-C₁₄ alkyl)dimethylbenzyl ammonium hydrogen sulfate, cetyl pyridinium bromide, and cetyl pyridinium hydrogen sulfide.

5. The composition according to claim 1, wherein the primary biocide is cetyl pyridinium chloride.

6. The composition according to claim 1, wherein the moisturizer component is chosen from urea, imidazolyl urea, hydantoin, dichlorodimethylhydantoin, bromochloro-dimethylhydantoin, dibromodimethylhydantoin, Glyceryl Triesters and biuret.

7. The composition according to claim 1, wherein the skin conditioner component is urea.

8. The composition according to claim 1, wherein the biocidal system comprises:
i) from about 75% to about 95% by weight of a primary biocide; and
ii) from about 5% to about 25% by weight of a pH buffer component.

9. The composition according to claim 1, wherein the biocidal system comprises:
i) from about 75% to about 95% by weight of cetyl pyridinium chloride; and
ii) from about 5% to about 25% by weight of pH buffer component.

10. The composition according to claim 1, wherein the nonionic surfactant is chosen from a polyoxyethylene C₆-C₁₂ alkylphenyl ether, polyoxyethylene sorbitan tri(C₁₂-C₁₈)-alkanoate, polyoxyethylene sorbitan di(C₁₂-C₁₈)-alkanoate, polyoxyethylene sorbitan mono(C₁₂-C₁₈)-alkanoate, or polyoxyethylene C₉-C₂₀ alkyl ether.

11. The composition according to claim 1, wherein the nonionic surfactant is a polyoxyethylene C₆-C₁₂ alkylphenyl ether having from about 8 to about 12 ethyleneoxy units.

12. The composition according to claim 1, wherein the cationic or ionic surfactant is a polyoxyethylene(5)isooctylphenyl ether, polyoxyethylene(8)isooctylphenyl ether, polyoxyethylene(9)nonylphenyl ether, polyoxyethylene(10)isooctylphenyl ether, polyoxyethylene(branched)nonylphenyl ether, polyoxyethylene(12)nonylphenyl ether, polyoxyethylene(12)isooctylphenyl ether, polyoxyethylene(40)nonylphenyl ether, and polyoxyethylene(40)isooctylphenyl ether.

12. The composition according to claim 1, wherein the cationic or ionic surfactant is polyethylene glycol 4-(1,1,3,3-tetramethylbutyl)phenyl ether.

13. The composition according to claim 1, wherein the cationic or ionic surfactant is a polyoxyethylene sorbitan mono-, di-, and tri-(C₁₂-C₁₈)-alkanoate.

14. The composition according to claim 1, wherein the cationic or ionic surfactant is a polyoxyethylene(20) sorbitan trioleate, polyoxyethylene(20) sorbitan monooleate, polyoxyethylene(20) sorbitan monostearate, polyoxyethylene(20) sorbitan monopalmitate, and polyoxyethylene(20) sorbitan monolaurate.

15. The composition according to claim 1, wherein the cationic or ionic surfactant is a polyoxyethylene C₉-C₂₀ alkyl ether.

16. The composition according to claim 1, wherein the cationic or ionic surfactant is a polyoxyethylene C₉-C₂₀ alkyl ether chosen from C₉-C₁₁ alkyl-(5)-ethoxylate, C₉-C₁₁ alkyl-(6)-ethoxylate, C₉-C₁₁ alkyl-(8)-ethoxylate, C₉-C₁₁ alkyl-(9)-ethoxylate, C₂-C₁₃ alkyl-(6.5)-ethoxylate, C₁₂-C₁₅ alkyl-(5)-ethoxylate, C₁₂-C₁₅ alkyl-(7)-ethoxylate, C₁₂-C₁₅ alkyl-(9)-ethoxylate, C₁₂-C₁₅ alkyl-(12)-ethoxylate, C₁₄-C₁₅ alkyl-(7)-ethoxylate, and C₁₁-C₁₅ alkyl-(7)-ethoxylate.

17. The composition according to claim 1, wherein the cationic or ionic surfactant has an HLB of from about 12 to about 18.

18. The composition according to claim 1, wherein the cationic or ionic surfactant has an HLB of from about 13 to about 16.

19. The composition according to claim 1, wherein the extradermal penetrating agent is a C₁-C₈ mono- or poly-hydroxy alcohol.

20. The composition according to claim 1, wherein the extradermal penetrating agent is chosen from benzyl alcohol, ethylene glycol, and propylene glycol.

21. The composition according to claim 1, wherein the extradermal penetrating agent is propylene glycol.

22. The composition according to claim 1, wherein the emollient base is chosen from C₃-C₁₄ linear or branched alkyl alcohols, C₃-C₁₄ linear or branched polyols, C₆-C₁₄ di-esters of C₆-C₁₂ diacids, hydrocarbons, natural waxes, vegetable oils, and silicones.

23. The composition according to claim 1, wherein the emollient base is a polyol having the formula:
HOCH₂—[CHOH]ₓ—CH₂OH
wherein the index x is from 1 to 6.

24. The composition according to claim 1, wherein the emollient base comprises a polyol chosen from glycerol, (2R,3R)-butane-1,2,3,4-tetraol, (2S,3R)-butane-1,2,3,4-tetraol, (2R,3S)-butane-1,2,3,4-tetraol, (2S,3S)-butane-1,2,3,4-tetraol, (2R,3R,4R)-pentane-1,2,3,4,5-pentaol, (2S,3R,4R)-pentane-1,2,3,4,5-pentaol, (2R,3S,4R)-pentane-1,2,3,4,5-pentaol, (2R,3R,4S)-pentane-1,2,3,4,5-pentaol, (2S,3S,4R)-pentane-1,2,3,4,5-pentaol, (2S,3R,4S)-pentane-1,2,3,4,5-pentaol, (2R,3S,4S)-pentane-1,2,3,4,5-pentaol, and (2S,3S,4S)-pentane-1,2,3,4,5-pentaol and poylglycols, polyethylene glycol, polyoxyethylene, polyethylene oxide, cyclomethicone and dimethicone.

25. The composition according to claim 1, wherein the emollient base is glycerol.

26. The composition according to claim 1, wherein the emollient base comprises glycerol and one or ethoxylated partial glyceride fatty acid esters or poylglycols, polyethylene glycol, polyoxyethylene, polyethylene oxide, cyclomethicone and dimethicone.

27. The composition according to claim 1, wherein the emollient base further comprises branched chain esters, ethoxylated partial glyceride fatty acid esters, protein derivatives, lanolin, lanolin derivatives, fatty alcohol ethoxylates, emollient oils, fatty acids, fatty alcohols, and fatty alcohol esters.

28. The composition according to claim 1, wherein the emollient base further comprises an emollient base chosen from isononyl isonanoate, dioctyl sebacate, isooctyl isooctanoate, dioctyl adipate, squalane, petrolatum, mineral oil, carnauba wax, candelilla wax, beeswax, sunflower oil, sesame oil, olive oil, cyclomethicone, or dimethicone.

29. The composition according to claim 1, wherein the emollient system comprises:
i) at least about 30% by weight of an extradermal penetrating agent; and
ii) at least about 60% by weight of an emollient base.

30. The composition according to claim 1, wherein the emollient system comprises:
i) from about 30% to about 40% by weight of an extradermal penetrating agent; and
ii) from about 60% to about 70% by weight of an emollient base.

31. The composition according to claim 1, wherein the thickening agent is chosen from hydroxynethyl cellulose, hydroxyethyl cellulose, methylcellulose, hydroxypropyl cellulose, methyl cellulose, carboxy methylcellulose, emulsifying waxes, alkyl triammonium methosulfate, and ceteraryl octanoate.

32. The composition according to claim 1, wherein the thickening agent is hydroxyethyl cellulose

33. The composition according to claim 1, wherein the thickening agent is chosen from, polysaccharides, linear sulfated polysaccharides.

34. The composition according to claim 1, wherein the thickening agent is polysaccharides and linear sulfated polysaccharides of natural origin, which increase the viscosity increase in solution.

35. The composition according to claim 1, wherein the thickening agent is xanthan gum.

36. The composition according to claim 1, wherein the thickening agent is a starch which can be unmodified or modified using acid, enzymes, alkaline, bleached, oxidized, acetylated, hydroxpropylated, octenylsuccinic anhydride, carboxyethylated, phosphate, hydroxypropyl, and acetylated oxidated), cationic, cold water, pregelatinized or instant starch.

37. A composition for improving teat and udder hygiene in a domesticated animal, comprising:
a) about 1.5% by weight of a biocidal system, comprising:
i) 90% by weight of cetyl pyridinium chloride; and
ii) 10% by weight of pH buffer;
b) about 0.2% by weight of polyoxyethylene(10)isooctylphenyl ether;
c) 0.1% to 5.0% of a skin conditioner
d) from about 1% to about 4% by weight of an emollient system comprising:
i) about 33.3% by weight of propylene glycol; and
ii) about 66.7% by weight of glycerol;
e) about 0.5% by weight of hydroxyethylcellulose; and
f) water.
